(19) United States
(12) Patent Application Publication
(10) Pub. No.: US 2005/0053645 A1
(43) Pub. Date: Mar. 10, 2005

(54) Adhesive transdermal formulations of diclofenac sodium

(76) Inventors: Giuseppe Passoni, Monza (IT); Paolo Casetta, Baveno (IT); Paola Stefanelli, Lecce (IT)

Correspondence Address: YOUNG & THOMPSON, 745 SOUTH 23RD STREET, 2ND FLOOR, ARLINGTON, VA, 22202, US

(21) Appl. No.: 10/500,159

(22) PCT Filed: Nov. 26, 2002
(86) PCT No.: PCT/EP02/13473

(30) Foreign Application Priority Data
Dec. 28, 2001 (IT) MI2001A002827

(51) Int. Cl. A61K 9/70; A61L 15/16

(52) U.S. Cl. 424/449

(57) ABSTRACT

An adhesive, transdermal formulation containing a suspension of diclofenac sodium in polyoxyl hydrogenated castor oil, a copolymer of aminoalkyl methacrylate and methacrylate, one or more crosslinking agents for the copolymer, an adhesion system and possibly other excipients.

The present invention concerns adhesive, transdermal formulations of diclofenac sodium and adhesive patches containing such formulations.

### BACKGROUND OF THE INVENTION

There are numerous known transdermal formulations in the form of adhesive patches able to release active ingredients of various kinds. This route of administration is particularly indicated for nonsteroid anti-inflammatory drugs, especially when prolonged treatments in specific areas of the body are required. Transdermal administration reduces the risk of side effects of such drugs, especially at a gastrointestinal level.

Diclofenac is one of the most commonly used nonsteroid anti-inflammatory drugs because of its marked pharmacological activity.

Transdermal formulations of diclofenac, particularly of its sodium salt, are described, for example, in patents No. EP 524582, EP 582727, U.S. Pat. No. 6,193,996, EP 209975, JP 6056660, WO 99/03461, U.S. Pat. No. 4,999,379 and EP 965626.

Some of the formulations described have been developed and are available on the market.

Most of the known adhesive formulations involve the solubilisation of the salt of diclofenac in solvent systems such as alcobols, water, glycols or mixtures of the same.

One disadvantage of such formulations lies in the fact that the active ingredient may be unevenly distributed because of possible precipitations of the drug due to seeds that may be present in the formulations and/or to uneven granulometry of the drug itself before its solution.

It has now been found that it is possible to formulate diclofenac sodium in an adhesive, transdermal composition by resorting to a suspension of the drug in polyoxyl hydrogenated castor oil. The use of a suspension overcomes the problems of stability and lack of homogeneity faced by the known formulations, while guaranteeing at least as much bioavailability, if not more.

### DESCRIPTION OF THE INVENTION

The subject of the present invention is an adhesive, transdermal formulation containing a suspension of diclofenac sodium in polyoxyl hydrogenated castor oil, a copolymer of aminoalkyl methacrylate and methacrylate, one or more crosslinking agents for said copolymer, an adhesion system and possibly other excipients.

The invention also concerns a skin patch constituted by a tissue on which the formulation is distributed and a protective layer.

### DETAILED DESCRIPTION OF THE INVENTION

The adhesive formulation according to the invention is characterised by the fact that the active ingredient is suspended in polyoxyl hydrogenated castor oil (see European Pharmacopoeia, 1997:1083), which is present at a percentage of between 5 and 50% of the total formulation, preferably between 10 and 30%.

The structural polymer that constitutes the matrix of the adhesive formulation of the invention is a cationic copolymer of a C₁-C₄ alkyl ester of methacrylic acid with an ester of methacrylic acid with a C₁-C₄ alcohol containing a secondary or tertiary amino group, with a mean molecular weight of between about 80,000 and about 500,000, preferably between about 100,000 and about 300,000.

In particular, a copolymer based on dimethylamino ethylmethacrylate and neutral esters of methacrylic acid, such as methyl, ethyl and butyl esters, is preferred.

Said copolymers are available on the market under the trademarks “Eudragit E 100”, “Eudragit E112.5”, “Plastoid E 35L, M or H”. The structural copolymer may typically constitute between approximately 10 and 30% in weight of the total adhesive formulation after drying, preferably between 12 and 25%.

Suitable crosslinking agents for the cationic copolymer are represented by polycarboxylic acids, in particular di- or tri-carboxylic acids such as succinic acid, adipic acid and fatty acids such as lauric acid or mixtures of the same.

The crosslinking agents may be present at percentages of between 1 and 20% in weight, again of the total weight of the adhesive formulation after drying.

The formulations of the invention may also contain agents such as an acidifiers/buffers, preservatives, flexibilizers.

The use of glycerine as a flexibilizer and citric acid as an acidifier is particularly preferred.

Finally, the formulation is completed with a suitable adhesive agent, preferably constituted by a copolymer of methacrylic acid with a C1-C4 ester of acrylic acid.

The adhesive formulations of the invention are prepared by mixing water, the crosslinking agents and the cationic copolymer. The mixture is heated to 70-80° C., agitated for a few hours, then cooled to 55-65° C. after which the flexibilizer is added.

Then, at the same temperature, a mixture of diclofenac sodium, polyoxyl hydrogenated castor oil, acidifier and water is added.

It is agitated, cooled and the adhesive polymer is added.

The formulation thus obtained is agitated, the pH adjusted to a value of between about 6 and 7, then it is spread on a suitable support, such as silicon paper, so as to give a quantity of diclofenac sodium of about 1 mg/cm².

It is then dried in a current of air at a temperature of between 40 and 120° C., and the support is applied to a suitable material, such as 100% non-woven polyester. The patch thus obtained is then packaged in a sachet that is impermeable to gas and liquids.

Hereafter are some examples of quantitative compositions of some formulations according to the invention, expressed as percentages in weight.

| | Examples of formulations | | | |
|---|---|---|---|---|
| | A | B | C | D |
| Diclofenac sodium | 17.7 | 17.7 | 17.7 | 17.7 |
| Acrylic copolymers | 32.9 | 33.1 | 37.3 | 42.1 |
| Carboxylic acids | 12.5 | 6.0 | 15.7 | 10.7 |
| Glycerol 30° Bé | 8.3 | 6.8 | 6.4 | 11.6 |
| Citric acid | 7.8 | 7.8 | 7.7 | 7.0 |
| Polyoxyl hydrogenated castor oil | 20.8 | 28.6 | 15.2 | 10.9 |

1. An adhesive, transdermal formulation containing a suspension of diclofenac sodium in polyoxyl hydrogenated castor oil, a cationic copolymer of a C₁-C₄ alkyl ester of methacrylic acid with an ester of methacrylic acid with a C₁-C₄ alcohol containing a secondary or tertiary amino group, one or more crosslinking agents for said copolymer, an adhesive system and, optionally, other excipients.

2. A formulation according to claim 1 wherein the crosslinking agents are chosen from polycarboxylic acids and long-chain acids.

3. A formulation according to claim 2 wherein the crosslinking agents are chosen from adipic acid, lauric acid, succinic acid.

4. A formulation according to claim 1, containing also acidifying/buffering agents, preservatives, flexibilizers.

5. A formulation according to claim 4 containing glycerin as a flexibilizer.

6. A formulation according to claim 4 containing citric acid as an acidifier.

7. A formulation according to claim 1, containing between 5 and 50% in weight of polyoxyl hydrogenated castor oil.

8. A formulation according to claim 1 containing between 10 and 30% of polyoxyl hydrogenated castor oil.

9. A formulation according to claim 1, wherein the cationic copolymer is a copolymer based on dimethylamino ethylmethacrylate and neutral esters of methacrylic acid.

10. A formulation according to claim 1 wherein the adhesive system is constituted by a copolymer of methacrylic acid with a C₁-C₄ ester of acrylic acid.

11. A transdermal patch constituted by a tissue on which a formulation according to claim 1 is spread and by a protective layer.

12. A transdermal patch according to claim 11 wherein the tissue is 100% non-woven polyester and the protective layer is silicon paper.

13. A transdermal patch according to claim 11, containing 1 mg of diclofenac sodium per cm² of patch.

14. A transdermal patch according to claim 12, containing 1 mg of diclofenac sodium per cm² of patch.
